(19) United States
(12) Patent Application Publication
(10) Pub. No.: US 2018/0333559 A1
(43) Pub. Date: Nov. 22, 2018

(54) CHEST TUBE POSITIONING DEVICE

(71) Applicant: Ian MAKEY, San Antonio, TX (US)

(72) Inventor: Ian MAKEY, San Antonio, TX (US)

(73) Assignee: THE BOARD OF REGENTS OF THE UNIVERSITY OF TEXAS SYSTEM, Austin, TX (US)

(21) Appl. No.: 15/559,644

(22) PCT Filed: Apr. 7, 2016
(86) PCT No.: PCT/US2016/026479

§ 371 Date: Sep. 19, 2017

Related U.S. Application Data
(60) Provisional application No. 62/144,107, filed on Apr. 7, 2015.

(51) Int. Cl. A61M 25/01; A61B 5/06; A61B 34/00; A61M 1/04

(52) U.S. Cl. CPC A61M 25/0127; A61B 5/062; A61B 2090/062; A61M 1/04; A61B 34/73

(57) ABSTRACT

The human chest cavity is lined with membranes referred to as the parietal pleura and the visceral pleura. The parietal pleura lines the chest cavity itself, while the viscera pleura is the membrane that lines the lungs. The space between the two membranes is called the intrapleural space or the pleural space. It normally has a small amount of fluid within it in a healthy individual. This fluid is drained and regulated by the lymphatic system and provides lubrication and cohesion between the pleura for normal lung function. Embodiments described herein are directed to a positioning device that can be inserted into a chest tube to facilitate positioning of the chest tube in a desired location for improve drainage of fluid from the chest space.

This Application claims priority to U.S. Provisional Patent Application Ser. No. 62/144,107 filed Apr. 9, 2015, which is incorporated herein by reference in its entirety.

### BACKGROUND

The human chest cavity is lined with membranes referred to as the parietal pleura and the visceral pleura. The parietal pleura lines the chest cavity itself, while the viscera pleura is the membrane that lines the lungs. The space between the two membranes is called the intrapleural space or the pleural space. It normally has a small amount of fluid within it in a healthy individual. This fluid is drained and regulated by the lymphatic system and provides lubrication and cohesion between the pleura for normal lung function.

An individual may accumulate air, fluid, or purulent drainage in the intrapleural space due to a number of pathologic conditions. When blood accumulates in this space, the condition is referred to as a hemothorax; air accumulation in the space is referred to as a pneumothorax; and purulent drainage accumulating in that space is referred to as empyema. Under such conditions as these, chest tubes may be required to provide drainage of air and excess fluid. Excess fluid in the intrapleural space may be caused by liver or kidney failure, congestive heart failure, infection, malignancy blocking the lymphatic system, trauma, or other injury to the lungs or chest cavity. If the amount of fluid is very small, chest tubes would not typically be necessary. However, if a considerable amount of fluid or blood is present, chest tubes and a drainage system will typically be required. Similar conditions may exist within the intrapleural space during and after surgical intervention into the chest cavity.

For trauma patients, a chest tube may be inserted in the field, in the emergency room or in the operating room. Unless an incision is made in the chest (thoracotomy) or a camera placed into the chest (thoracoscopy), it is difficult to guide the chest tube to the desired location in the chest.

### SUMMARY

Certain embodiments are directed to a chest tube positioning device. The device can consists of two separate parts: (i) an internal component and (ii) an external component. The internal component is an elongated member having an external diameter that is smaller than the internal diameter of the chest tube, enabling its insertion inside the lumen of the chest tube. The distal end of the internal component is comprised of 1, 2, 3, 4, 5, 6, 7, 8, 9, 10, 11, 12, or more metal or magnetic elements (guidance elements), which are configured to be influenced by or attracted to the external component. The internal component can be manipulated using the external component to facilitate positioning of the chest tube to a desired location in order to improve drainage of fluid from the chest space. In certain aspects the internal and external components can be supplied together or as individual components.

The external component or guidance device comprises a magnetic or magnetic field generating portion and an optional handle. In certain aspects the guidance device can be an electromagnet or a solenoid. As used herein a solenoid is a coil wound into a tightly packed helix. In certain aspects the solenoid can be configured such that a part of a subject's body (e.g., shoulder, neck, chest, etc.) can be positioned in the lumen of the solenoid. In certain aspects the guidance device can be position externally at an appropriate position so as to draw the internal component to a target location within the chest. For example the guidance device can be position over a subject's shoulder, chest, or back with the target region between the internal component and the guidance device, where the guidance device is active and draws the internal component to the target region. In a further aspect, the external component can be configured to be moveable over a chest wall. In certain embodiments the external component is configured with wheels or rollers to facilitate movement over the patient's body. In a certain embodiments the external component will have a slippery or low friction surface to facilitate movement over the patient's body. In other embodiments the external component or guidance device will have a smooth surface that can be moved across skin with minimal friction. In a certain aspect the guidance is configured as a disc or short cylinder that can be manipulated by hand. In certain respects the external component has a polarity that complements or matches the polarity of and attracts or repels, respectively, the distal end of the internal component. In certain aspects the magnetic portion of the external component is configured to provide a regulatable magnetic field in which the attraction or repulsion between the external component and distal end of the internal component can be increased or decreased as needed when altering the position of the distal end of the chest tube.

In certain aspects, the internal component is removable, reusable, and sterilizable. The internal component can be about 25, 30, 35, 40, 45, 50, 55, or 60 centimeters in length, including all values and ranges there between. The internal component is configured to be long enough to, or be attached to an extension that allows the internal guidance component to reach the distal end of a chest tube with a proximal end configured so the internal component can be removed once the chest tube is in position. In certain embodiment the internal component is a solid or segmented (partially or along the length) flexible elongated cylinder having a guidance portion located in the distal half. In certain aspects the internal component can be a distal guidance tip removeably or permanently connected to a more rigid proximal portion. In a further embodiment the internal component forms a lumen or fluid channel. The internal component lumen or fluid channel, if present, is configured to allow fluid and air to pass through or along the internal component allowing removal of fluid and/or air while the internal component is being advanced to the distal end of the chest tube or during positioning of the chest tube. In certain aspects the guidance portion is located in the distal 2, 4, 6, 8, 10, 12, 14, 16, 18, or 20 centimeters of the internal component. The guidance portion can be 5, 6, 7, 8, 9, 10, 11, 12 centimeters, including all values and ranges there between. In certain respects the guidance portion can comprise 1, 2, 3, 4, 5, 6, 7, 8, 9, 10, 11, 12 guidance elements, e.g., magnets or magnetizable material, positioned along the distal portion of the internal component with a spacing of 0.1, 0.5, 1, 2, 4, 6, 8, 10 mm or cm between each consecutive guidance elements, including all values and ranges there between. In certain aspects the guidance elements can be spherical, cylindrical, disc, square, or rectangular shaped elements and can be used in various combinations. The guidance elements are combined, spaced, positioned, shaped, and sized in such a way as to provide flexibility to the guidance portion of the internal component. In certain aspects the shape and diameter of the guidance elements are provided in an alternating configuration to provide flexibility, e.g., alternating cylindrical and spherical guidance elements (see FIG. 5 for an example). In certain respects a series of guidance elements can incorporate non-magnetizable elements as well to provide flexibility for the guidance portion of the internal component. In certain aspect the distal end of the internal component can be advanced to the distal end of the chest tube were it is manipulated by the external component. In certain aspects the internal component is equipped with a stop mechanism that stops it from being advanced beyond a certain distance. In certain aspects the stop mechanism is configured with markers on the internal component to monitor the distance of insertion. In certain aspects the stop mechanism is adjustable. The stop mechanism can be moveably positioned with in the proximal 2, 4, 6, 8, 10, 12, or 14 centimeters of the internal component.

In the following discussion, the terms “proximal” and “distal” will be used to describe opposing ends of the chest tube or the internal component. The term “proximal” is used in its conventional sense to refer to the end of the chest tube system or internal component that is closest to the operator during use. The term “distal” is used in its conventional sense to refer to the end of the chest tube or internal component that is initially inserted into the patient or that is closest to the patient during use.

Other embodiments of the invention are discussed throughout this application. Any embodiment discussed with respect to one aspect of the invention applies to other aspects of the invention as well and vice versa. Each embodiment described herein is understood to be embodiments of the invention that are applicable to all aspects of the invention. It is contemplated that any embodiment discussed herein can be implemented with respect to any method or composition of the invention, and vice versa. Furthermore, compositions and kits of the invention can be used to achieve the methods of the invention.

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF THE DRAWINGS

The following drawings form part of the present specification and are included to further demonstrate certain aspects of the present invention. The invention may be better understood by reference to one or more of these drawings in combination with the detailed description of the specification embodiments presented herein.

FIG. 1 illustrates one embodiment of the positioning device and its use in guiding or positioning a chest tube in a patient.

FIG. 2 illustrates other embodiments of the guidance portion of the internal component including an internal component forming a lumen and a solid internal component having multiple guidance elements.

FIG. 3 illustrates an embodiment incorporating a stop mechanism in the proximal portion of the internal component.

FIG. 4 is a photograph of one embodiment of the (A) internal component and (B) external component of a positioning device.

FIG. 5 is an illustration of one embodiment of an arrangement of guidance elements comprising alternating cylinders and spheres.

### DESCRIPTION

The positioning device described herein facilitates positioning of the chest tube to the desired location in the chest in order to help evacuation of blood or fluid. Use of the positioning device would preclude having to place a second chest tube or even surgery. In certain embodiments the positioning device is configured to be sterilized and can be reused.

Since it is a re-usable, the positioning device is cost-effective.

Of trauma patients that require a chest tube, approximately 4-8% have blood that remains in the chest space, i.e., a post-traumatic retained hemothorax. Depending on the size of the retained hemothorax, additional procedures are required to evacuate the blood. Additional procedures include insertion of a second chest tube, administration of clot busting drugs, video-assisted thorascopic surgery, or thoracotomy. The positioning device described herein provides a solution for removal of blood or other fluids without resorting to additional procedures.

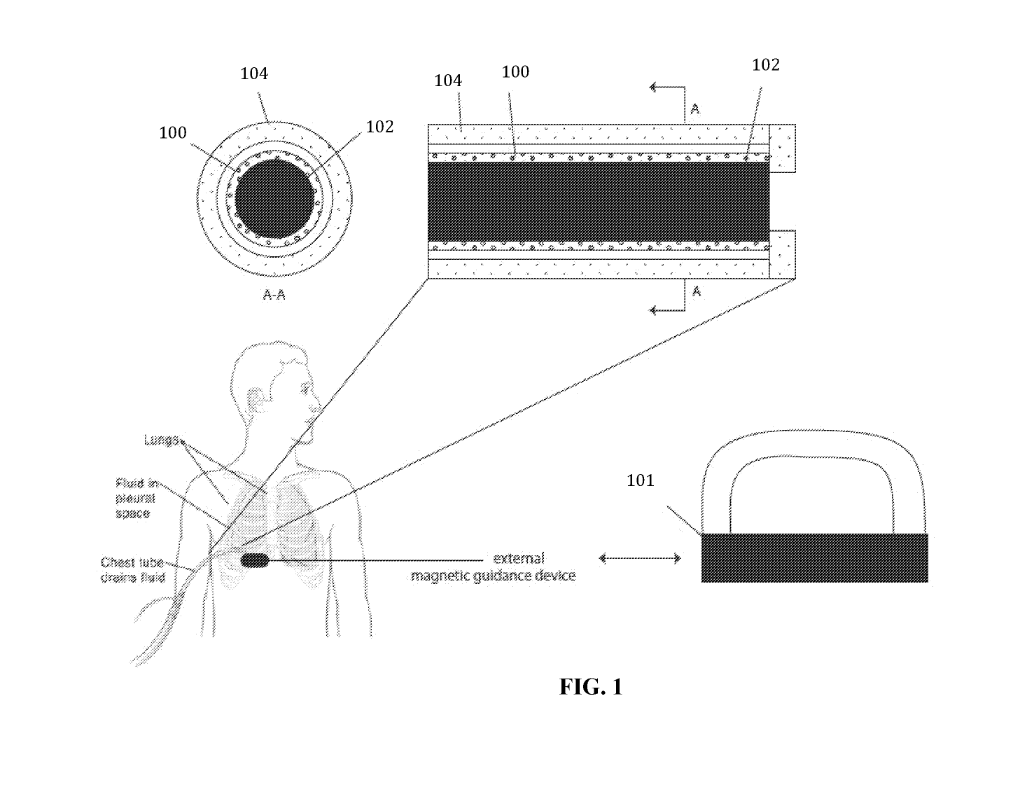

In certain embodiments the internal component will comprise an elongated cylinder or tube having guidance elements incorporated in the distal end forming a guidance portion. The internal component can have a regular or irregular cross-section, including but not limited to a circle, oval, square, pentagon, hexagon, octagon, or other polygon. FIG. 1 illustrates one embodiment of the positioning device. FIG. 1 shows the insertion of a chest tube in a patient. The chest tube in this picture has inserted within its lumen an internal component or guidance component. FIG. 1 includes a magnified image of the distal portion of the internal component. The magnified portion shows chest tube 104 containing internal component 100 having a solid guidance element 102. Guidance element 102 can be manipulated using the external component 101, which comprises a magnetic portion and an optional handle so that the external component can be moved along the chest of the patient to position the chest tube by influencing or attracting or repulsing the guidance portion of the internal component located in the chest tube. In certain aspects the guidance element(s) are located in the distal end of the tube. In certain aspects the internal component is about 20, 25, 30, 35 to 30, 35, 40 cm in length, including all lengths and ranges there between. The internal component can have an outer diameter of approximately 18, 19, 20, 22, 24, 26, 28, 30, 32, to 34 French or about 6 to 12 mm. In further aspects the internal component is made from flexible plastic, such as soft polyurethane or silicon (e.g., PEBAX®, PELLETHANE®, CARBOTHANE®, having a hardness of about 75A to 55D. The guidance portion of the internal component can be enclosed by plastic or other medically compatible coatings.

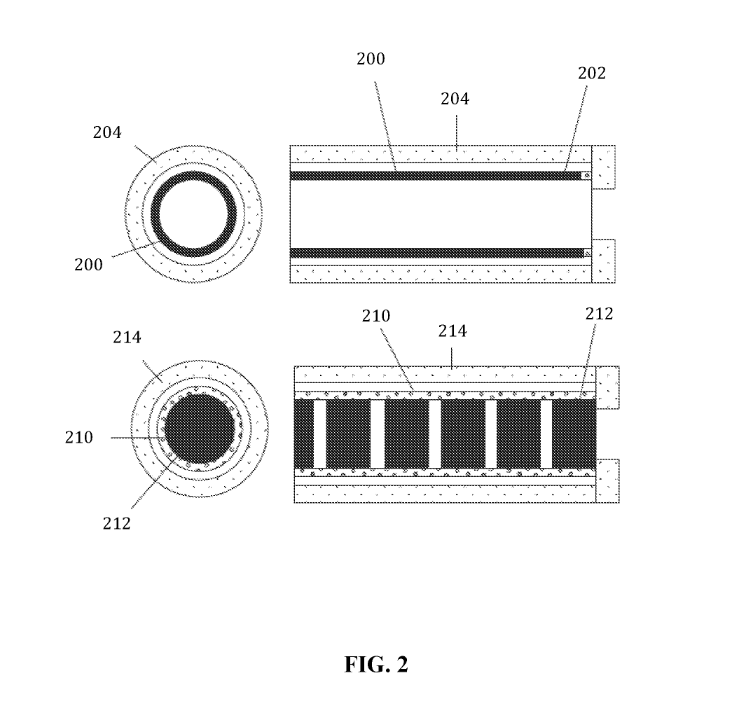

FIG. 2 illustrates other embodiments that use a tube configuration or multiple solid guidance elements (stacked guidance elements). In certain respects the guidance elements are stacked to approximate a longer guidance portion yet allowing flexibility within the guidance portion. Distal portions of chest tube 204 and 214 are shown with internal component 200 and 210 having guidance elements 202 or 212.

Guidance elements can comprise a metal, magnetizable, or magnetic substrate comprising a metal and/or metal alloy. As used herein “magnetic substrate” refers to both magnets and magnetically attractive materials. As used herein “magnet” refers to a material that both produces its own magnetic field and responds to magnetic fields. Magnets include permanent magnets, which remain magnetized, and impermanent magnets, which lose their memory of previous magnetizations, or electromagnets that can be magnetized by electric current, which can be modulated to adjust the strength of the magnet. The magnetic portion of the external component can be configured as an adjustable electromagnets. Magnets include but are not limited to neodymium, samarium cobalt, ceramic (Ferrite), and Alnico (Aluminum Nickel Cobalt). In certain aspects the guidance elements are embedded in a polymer. As used herein “magnetically attractive material” refers to materials that do not produce a magnetic field, but that are attracted to a magnetic field or to each other when in the presence of a magnetic field, and include paramagnetic materials. Magnetically attractive materials include but are not limited to iron coated with material to make it biocompatible, and steel. In certain aspects the guidance element can comprise a neodinium iron boron magnetic substrate. In one aspect the magnetic substrate is configured as cylinder or a series of rings. In other aspects the magnetic substrate can be beads or rods. In certain embodiments the polarity of the guidance element(s) is configured to be compatible with the magnetic portion of the external component.

A magnet is a material or object that produces a magnetic field. This magnetic field is invisible but is responsible for the most notable property of a magnet: a force that pulls on other ferromagnetic materials, such as iron, and attracts or repels other magnets. A permanent magnet is an object made from a material that is magnetized and creates its own persistent magnetic field. These include iron, nickel, cobalt, some alloys of rare earth metals, and some naturally occurring minerals such as lodestone. An electromagnet is made from a coil of wire that acts as a magnet when an electric current passes through it but stops being a magnet when the current stops. Often, the coil is wrapped around a core of ferromagnetic material such as steel, which greatly enhances the magnetic field produced by the coil.

The overall strength of a magnet or magnetic field is measured by its magnetic moment or, alternatively, the total magnetic flux it produces. The strength of a given magnet is sometimes given in terms of its pull force—its ability to move (push/pull) other objects. The pull force exerted by either an electromagnet or a permanent magnet at the “air gap” (i.e., the point in space where the magnet ends) is given by the Maxwell equation: F=B²A/2 μ₀, where F is force (SI unit: newton), A is the cross section of the area of the pole in square meters, B is the magnetic induction exerted by the magnet, μ is the permeability of the intervening medium (SI unit: newton per ampere squared). Classically, the force between two magnetic poles is given by: F=μqₘ₁qₘ₂/4πr² where F is force (SI unit: newton), qₘ₁ and qₘ₂ are the magnitudes of magnetic poles (SI unit: ampere-meter), μ is the permeability of the intervening medium (SI unit: newton per ampere squared), r is the separation (SI unit: meter).

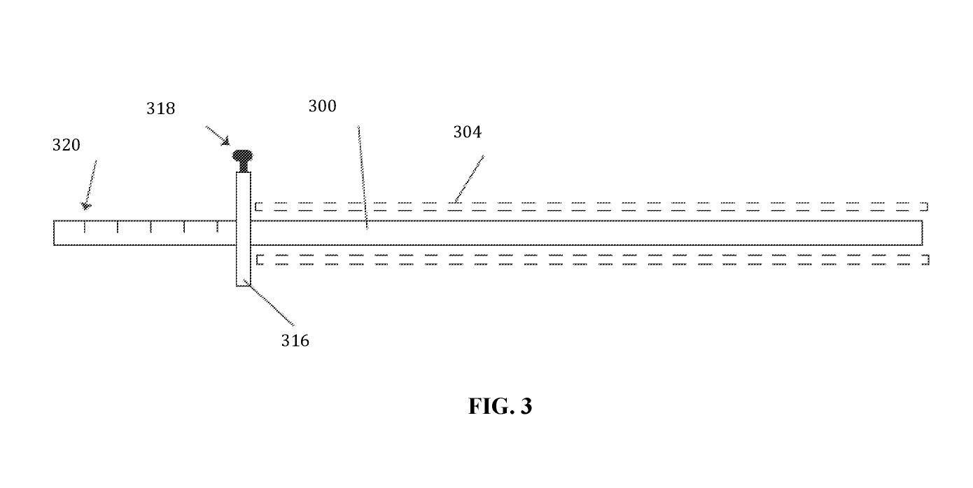

In certain aspects an internal component is configured to be placed within a chest tube prior to, during, or after chest tube insertion. Typically the internal component will be sterile and in a package or container that will maintain sterility until use. The distal end of the internal component can be advanced to the distal end of the chest tube, but preferably not past the distal end of the chest tube. FIG. 3 illustrates one embodiment of an internal component that incorporates stop mechanism 316. FIG. 3 shows internal component 300 inserted into chest tube 304. Stop mechanism 316 is located at about the proximal end of internal component 300. Distance markers 320 can be included to monitor the depth of insertion of internal component 300. The stop mechanism is configured so that it can be locked into position at a certain depth. The stop mechanism 316 can be moveable along internal component 300 and can be locked in position using locking mechanism 318. In certain aspects stop mechanism 316 can be adjusted to a depth indicated by distance marker 320. An external component is used to influence, attract or otherwise position the distal end of the internal component/chest tube in the patient's chest. The internal component is designed so that the guidance elements are influenced by the external component and enable the movement of the chest tube to the desired location. In certain aspects the external component is a handheld device manipulated by the physician or other person. The external component can be moved along the patient's chest to move the chest tube within the patient's chest.

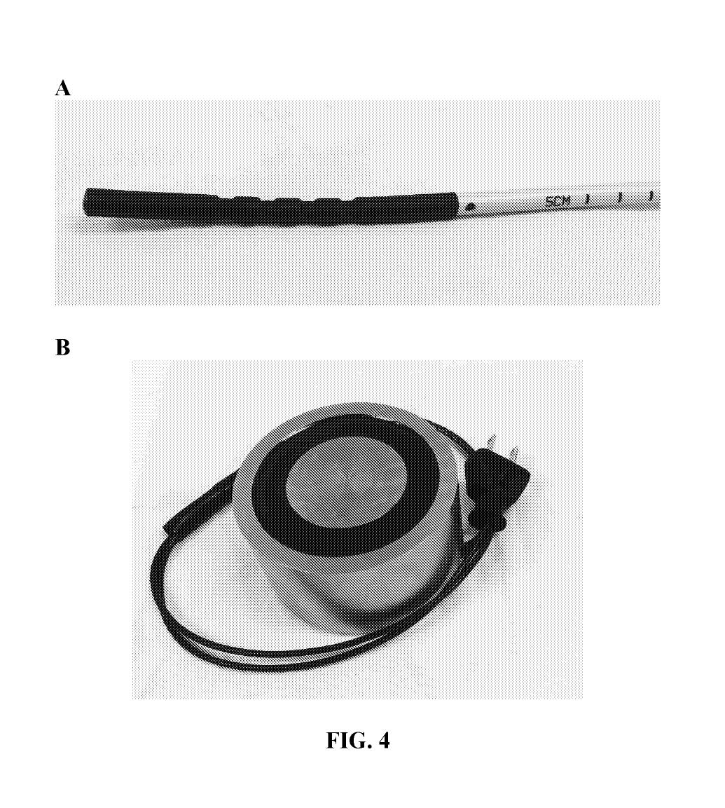

FIG. 4 is a photograph of one embodiment of an internal guidance device as described herein. FIG. 4A shows a guidance portion of an internal component in black that contains 9 guidance elements. The distal and proximal guidance element being cylindrical and the intermediate 7 guidance element alternating from spherical to cylindrical. The guidance portion is connected to a second portion that, as shown, has depth gauge markings starting at 5 cm. FIG. 4B shows one embodiment of an external guidance component comprising a disk shaped device configured to provide an adjustable magnetic field. In other embodiments the external guidance component can comprise a solenoid of similar device to produce a magnetic field.

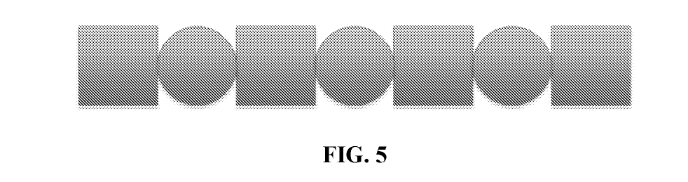

FIG. 5 illustrates one non-limiting embodiment showing one particular configuration for an alternating cylinder/sphere arrangement of guidance elements. The elements can be covered in a flexible membrane that allows the distal end of the internal component to bend in order to more easily follow the path of the external magnetic field.

The location of retained blood, fluid, or air can be determined using a variety of imaging techniques, including but not limited to X-ray, sonogram, or CAT scans. Once the location is determined the chest tube is moved to the location with the retained blood. In certain aspects, once the chest tube is in place the internal component is removed and the chest tube can placed back to the pleuravac system. The internal component is washed, sterilized, and stored until next use.

In certain embodiments the external component will be configured such that the strength of the magnetic field can be modulated. Thus the strength of the external component can be adjusted to achieve the desired “attraction” or to apply a sufficient force to move the chest tube. In certain aspects the external component is a handheld magnetic device that is configured to be placed against the patient's chest wall and, optionally moved manually to adjust the position of the distal end of the chest tube located in the patient's chest. In certain aspects the external guidance component can be about 2 to 24 inches long and configured to be placed or held on a subject's shoulder, chest, or back.

In one aspect, embodiments related to chest tubes used to drain fluids (gas or liquids) from pleural cavities. Chest tubes in accordance with embodiments of the invention include features that can be deployed with an internal component or an internal component can be inserted into a deployed chest tube. In another aspect, embodiments relate to methods for using chest tubes with an internal component to drain fluids from pleural cavities. Methods in accordance with embodiments described herein include deploying, after or concurrently with insertion of the chest tubes, the internal component. In certain aspects a variety of positioning devices can be supplied as kit and include internal components of various lengths, external diameters, and configurations to be operable with any type of chest tube.

While the invention has been described with respect to a limited number of embodiments, those skilled in the art, having benefit of this disclosure, will appreciate that other embodiments can be devised which do not depart from the scope of the invention as disclosed herein. Accordingly, the scope of the invention should be limited only by the attached claims.

1. A chest tube positioning device having an elongated body configured to be inserted in the chest tube lumen, the body having a proximal and a distal portion, the distal portion including at least one guidance element forming a guidance portion that is configured to interact or be influenced by an external guiding force.

2. The device of claim 1, wherein the external force is a magnetic force.

3. The device of claim 1, configured to permit a physician or trained user to position the chest tube in a desired location within the chest.

4. The device of claim 1, wherein the guidance portion comprises 2 to 20 guidance elements.

5. The device of claim 1, wherein the guidance element is cylindrical, spherical, or a combination thereof.

6. The device of claim 1, wherein the guidance element is comprised of a magnetic or magnetizable material.

7. The device of claim 1, wherein the length of the elongated body is between or equal to 20, 25, 30, 35 to 30, 35, or 40 cm and the diameter is about 18 to 34 French.

8. The device of claim 7, wherein the guidance portion can have a variable diameter that ranges from 18 to 34.

9. The device of claim 1, further comprising a stop mechanism coupled to the proximal portion of the device.

10. The device of claim 1, further comprising one or more distance markers coupled to or incorporated into or onto the device, wherein the distance markers correlate to an insertion depth.

11. A chest tube position system comprising the device of claim 1 and an external guidance component configured to produce a modulatable external force that influences the position of the internal guidance component when in use.

12. The system of claim 11, wherein the external component produces a regulateable magnetic field.
